(19) United States
(12) Patent Application Publication
(10) Pub. No.: US 2002/0099002 A1
(43) Pub. Date: Jul. 25, 2002

(54) Inhibition of angiogenesis by verotoxins

(75) Inventors: Sara Arab, North York (CA); Clifford A. Lingwood, Toronto (CA)

Correspondence Address: LAHIVE & COCKFIELD, 28 STATE STREET, BOSTON, MA, 02109, US

(73) Assignee: Hospital for Sick Children Research and Development Limited Partnership at Reel 010228

(21) Appl. No.: 09/877,399

(22) Filed: Jun. 8, 2001

Related U.S. Application Data
(63) Continuation of application No. 09/080,027, filed on May 15, 1998, now abandoned.
(60) Provisional application No. 60/046,640, filed on May 16, 1997.

(51) Int. Cl. A61K 38/16

(52) U.S. Cl. 514/2

(57) ABSTRACT

The invention pertains to methods for inhibiting angiogenesis. Diagnostic and therapeutic methods utilizing anti-angiogenic agents which bind Gb3 or CD77, e.g., verotoxins, are provided. Methods for treating multiple drug resistant tumors are also provided.

### RELATED APPLICATION

This application claims priority to the U.S. Provisional Application Serial No. 60/046,640 entitled “Inhibition of Angiogenesis by Verotoxin” filed May 16, 1997.

### BACKGROUND OF THE INVENTION

Primary tumor growth and the formation of metastasis depend on the process of angiogenesis, the establishment of new blood vessels from preexisting ones. The induction of angiogenesis is most likely to occur during the early stages of tumor development. This process is regulated both by several inducers and inhibitors of endothelial cell proliferation and migration.

Inhibition of angiogenesis and targeting of the tumor vasculature are highly effective in controlling tumor growth. Accordingly, targeting angiogenic and angiostatic processes by using angiogenesis inhibitors, receptor antagonists, and antibodies are important therapeutic tools in angiogenic diseases.

### SUMMARY OF THE INVENTION

Methods of the invention are based in part on the discovery that cells required for the growth and development of blood vessels, e.g., endothelial cells, bear Gb₃ receptors.

Accordingly, the present invention pertains to methods for inhibiting angiogenesis. These methods include administering an effective amount of an anti-angiogenic agent that binds Gb₃, such that angiogenesis is inhibited.

In one aspect of the invention, the tissue is a tumor. In another aspect of the invention, the tumor is a cancer, e.g., ovarian cancer, testicular cancer, or breast cancer.

The anti-angiogenic agent is an agent capable of binding Gb₃ and inhibiting the establishment of new blood vessels in tumor growth. Anti-antigenic agents include a verotoxin, e.g., verotoxin 1, verotoxin 1 B-subunit, verotoxin 2, or verotoxin 2c. Anti-angiogenic agents also include agents capable of binding Gb₃ which are linked to toxins capable of inhibiting the establishment of new blood vessels in tumor growth, e.g., PagG adhesin or antibodies to Gb₃ which are linked toxins, e.g., ricin.

The present invention further pertains to methods for treating drug-resistant tumors. These methods include administering to a subject in need thereof an effective amount of a verotoxin, such that the drug-resistant tumor is treated.

In another aspect of the invention, the drug-resistant tumor is a cancer, e.g., ovarian cancer, testicular cancer, or breast cancer.

In another aspect of the invention, the drug-resistant tumor is treated with an effective amount of verotoxin 1, verotoxin 1 B-subunit, verotoxin 2, or verotoxin 2c.

In another aspect of the invention, the effective amount of a verotoxin is administered in a pharmaceutically acceptable carrier.

The invention further pertains to a method for visualizing a blood vessel. Such methods involve contacting the blood vessel with a verotoxin such that the verotoxin binds to the blood vessel and visualizing the verotoxin bound to the blood vessel such that the blood vessel is visualized.

In another aspect of the invention, the visualizing step includes contacting the bound verotoxin with a verotoxin-binding antibody.

In another aspect of the invention, the verotoxin-binding antibody is labeled, e.g., FITC labeled verotoxin-binding antibody.

In another aspect of the invention, the blood vessel to be visualized is contacted with verotoxin 1, verotoxin 1 B-subunit, verotoxin 2, or verotoxin 2c.

The invention further pertains to a method for determining whether a tumor is multi-drug resistant. These methods include determining the amount of Gb₃ in a tumor sample, and comparing the amount of Gb₃ in the tumor sample with a preselected value, thereby determining whether the tumor is multi-drug resistant.

### DETAILED DESCRIPTION OF THE INVENTION

The present invention provides a method for inhibiting angiogenesis, e.g., tumor-induced angiogenesis which is caused by disorders characterized by the abnormal proliferation of cells or tumor-induced angiogenesis. These disorders include cancer, e.g., ovarian cancer, testicular cancer, breast cancer, as well as, other non-cancer related disorders, e.g., atherosclerosis, ischemic heart diseases, and inflammation.

Another aspect of the invention pertains to methods for treating a subject, e.g., a human, having a disease or disorder characterized by (or associated with) angiogenesis, e.g., tumor-induced angiogenesis, e.g., cancer, e.g., ovarian cancer, testicular cancer, breast cancer. These methods include the step of administering the anti-angiogenic agent of this invention capable of inhibiting angiogenesis to the subject such that treatment occurs. Non-limiting examples of disorders or diseases characterized by tumor-induced angiogenesis include cancer, e.g., ovarian cancer, testicular cancer, breast cancer.

The terms “treating” or “treatment”, as used herein, refer to reduction or alleviation of at least one adverse effect or symptom of a disorder or disease, e.g., a disorder or disease characterized by or associated with angiogenesis.

The method comprises administering an effective amount of an anti-angiogenic agent that binds Gb₃ (also designated CD77) such that angiogenesis is inhibited. The anti-angiogenic agents bind to cells which are involved in the growth and development of blood vessels and bear a Gb₃ receptor, e.g., endothelial cells. Anti-angiogenic agents are capable of inhibiting angiogenesis by a variety of mechanisms. For example, an anti-angiogenic agent can bind the cell via the Gb₃ receptor and induce cell death. Alternatively, anti-angiogenic agents can bind the cell via the Gb₃ receptor, become incorporated into the cell thus inducing cell death (e.g., by induction of apoptosis).

Preferred anti-angiogenic agents include, among others, a verotoxin (VT). VTs, also known as SHIGA-like toxins, comprise a family known as VT 1, VT2, VT2c, and VT2e of subunit toxins elaborated by some strains of E. coli. Cell toxicity is mediated via the binding of the B subunit of the holotoxin to Gb₃. VTs are described in U.S. patent application Ser. No. 08/563,394, entitled “Verotoxin Pharmaceutical Compositions and Medical Treatments Therewith”, filed Nov. 28, 1995, which is hereby incorporated by reference.

The isolation and purification of VTs have been earlier described. VT1 can be prepared genetically from the high expression recombinant E. coli pJB28 (J. Bacteriol. 166:375 and 169:4313) and purified by protein purification procedures (FEMS Microbiol. Lett. 41:63). VT2 can be obtained from R82 (Infect. Immun. 56:1926-1933 (1988)) and purified by protein purification procedures (FEMS Microbiol. Lett. 48:379-383 (1987)). VT2c can be obtained from clinical strain E32511 and purified by protein purification procedures (FEMS Microbiol. Lett. 51:211-216 (1988)). VT1 B subunit can be prepared according to Ramatour, et al. Biochem. J. 272:805-811 (1990).

The VTs consist of a 30 kDa enzymatic subunit which is capable of inhibiting protein synthesis. The A subunit is noncovalently associated with a pentameric 7kDa B subunit array which binds to Gb₃. In addition to the cytotoxic effects of VTs on a wide range of cells by the A subunit inhibition of protein synthesis, VT1, and the receptor binding subunit alone, also induce morphological changes and DNA fragmentation characteristic of apoptosis in Gb₃-positive cells.

Cell binding of the VT1 B subunit alone can induce apoptosis in B cells and Gb₃ containing B cells are prone to apoptosis during B-cell differentiation. Sensitivity to VT1 is a function of cell cycle and cells at GI/S boundary are particularly sensitive while stationary phase cells are refractory. Once internalized by receptor mediated endocytosis, Gb₃-bound VT1 can follow a unique pathway of intracellular retrograde transport to the Golgi/ER and nuclear membrane. Gb₃ binding is involved in α-interferon receptor function, and in CD 19 signal transduction in germinal center B cells to mediate homotypic adhesion and apoptosis.

Additional examples of anti-angiogenic agents include, among others, PagG adhesin (Kihlberg, et al. J. Am. Chem. Soc. 111:6364-6368 (1989) and antibodies to Gb₃ or CD77 which can be linked to a toxin capable of inhibiting angiogenesis. Antibodies can be polyclonal, or more preferably, monoclonal. An intact antibody, or a fragment thereof (e.g., Fab or F(ab′)₂) can be used. A variety of monoclonal antibodies to Gb3 or CD77 are discussed in Oosterwijk, et al. (1991) Int. J. Cancer 48(6):848-854; Kasai, et al. (1985) J. Immunogenet. 12(4-5):213-220; and Pallensen, et al. (1987) J. Cancer Res. Clin. Oncol. 113(1):78-86, the contents of which are incorporated by reference herein. Also, anti-Gb₃ is commercially available (AN 1003566, Biodesign International, Kennebunkport, Me., USA). Toxins which can be linked to these antibodies include, among others, VTs, and other immunotoxins known in the art, e.g., ricin.

The anti-angiogenic agents may be administered to the subject by methods well-known in the art, namely, intravenously, intra-arterially, topically, subcutaneously, by ingestion, intra-muscular injection, inhalation, and the like, as is appropriately suitable to the disease. For treatment of a skin cancer, subcutaneous application is preferred.

The VT or its B subunit is typically administered in a suitable vehicle in which the active VT or B subunit ingredient is either dissolved or suspended in a liquid, such as serum to permit the VT to be delivered, for example, in one aspect from the bloodstream or in the alternative aspect subcutaneously to the cells. Alternatively, for example, solutions are typically alcohol solutions, dimethyl sulfoxide solutions, or aqueous solutions containing, for example, polyethylene glycol containing, for example, polyethylene glycol 400, Cremophor-EL, or Cyclodextrin. Such vehicles are well-known in the art and useful for the purpose of delivering a pharmaceutical to the site of action.

The invention further pertains to the treatment of drug-resistant tumors. The method includes administering to a subject in need thereof an effective amount of an anti-angiogenic agent, e.g., verotoxin, such that the drug-resistant tumor is treated.

Drug-resistant tumors have been found to contain high levels of Gb₃. Accordingly, agents capable of binding to Gb₃ and inhibiting the establishment of new blood vessels in tumor growth can be used for the treatment of drug-resistant tumors. Anti-angiogenic agents include a VT, e.g., VT 1, VT 1 B-subunit, VT 2, or VT 2c. Anti-angiogenic agents also include agents capable of binding Gb₃ which are linked to toxins capable of inhibiting the establishment of new blood vessels in tumor growth, e.g., PagG adhesin or antibodies to Gb₃ which are linked toxins, e.g., VTs or ricin.

The invention further provides a method for detecting the presence of a disorder characterized by abnormal cell proliferation, e.g., tumor-induced angiogenesis, e.g., cancer, e.g., ovarian cancer, testicular cancer, breast cancer. The method involves contacting the biological sample, e.g., tissue sample, with a compound or an agent capable of detecting Gb₃, e.g., fluorescently labeled VT1, determining the amount of Gb₃ expressed in the sample, comparing the amount of Gb₃ expressed in the sample to a control sample, and forming a diagnosis based on the amount of Gb₃ expressed in the sample compared to the control sample. A preferred agent for detecting Gb₃ is a labeled or labelable probe capable of hybridizing to Gb₃. The probe can be, for example, FITC VT1. A preferred agent for detecting Gb₃ is a labeled or labelable antibody capable of binding to Gb₃. Antibodies can be polyclonal, or more preferably, monoclonal. An intact antibody, or a fragment thereof (e.g., Fab or F(ab′)₂) can be used. The term “labeled or labelable”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect Gb₃ in a biological sample in vitro as well as in vivo. For example, in vitro techniques for detection of Gb₃include enzyme linked immunosorbent assays (ELISAs), Western blots, immunoprecipitations and immunofluorescence. Alternatively, Gb₃ can be detected in vivo in a subject by introducing into the subject a labeled anti-Gb₃ antibody. For example, the antibody can be labeled with a radioactive marker whose presence and location in a subject can be detected by standard imaging techniques.

The invention further provides a method for monitoring a previously diagnosed subject with a disorder characterized by abnormal cell proliferation, e.g., tumor-induced angiogenesis, e.g., cancer, e.g., ovarian cancer, testicular cancer, breast cancer. The method involves contacting a biological sample, e.g., a tissue sample, from the subject with an agent capable of detecting Gb₃, e.g., fluorescently labeled VT1, determining the amount of Gb₃ expressed in the sample, comparing the amount of Gb₃ expressed in the sample to a the amount of Gb₃ expressed in a sample previously obtained from the same subject to determine the progression of the disease, e.g., measuring the increase or decrease in levels of Gb₃ over time in a subject.

The invention further pertains to a method for visualizing a blood vessel. This method includes contacting the blood vessel with a VT such that VT binds to the blood vessel, and visualizing VT bound to the blood vessel, such that the blood vessel is visualized. Preferred agents for visualization include a VT-binding antibody, a VT-binding antibody which is labeled, e.g., with FITC. Preferred VTs include VT1, VT1 B-subunit, VT2, and VT2c.

The invention further pertains to a method for determining whether a tumor is multi-drug resistant. The method includes determining the amount of Gb₃ in a tumor sample, comparing the amount of Gb₃ in the tumor sample with a preselected value, thereby determining whether the tumor is multi-drug resistant.

The following invention is further illustrated by the following examples, which should not be construed as further limiting. The contents of all references, pending patent applications and published patents, cited throughout this application are hereby expressly incorporated by reference.

### EXAMPLES

Materials:

Goat anti mouse IgG, was from Sigma, OCT compound from Miles laboratories, PHI monoclonal anti-VT1 was from our laboratory [Boulanger, J. et al., J Clin Micro. 28:2830-2833 (1990)]. VT1 B subunit [Ramotar, K. et al., Biochem. J. 272:805-811 (1990)] was prepared as described below and FITC labeled as previously [Lingwood, C. A., Nephron. 66:21-28 (1994)]. DABCO (1,4-Diazabicyclo(2,2,2)-Octane) was from Sigma. Fresh surgically removed primary ovarian tumors and metastases were stored at −70° C. prior to analyses. For frozen sections, a separate sample was snap frozen in embedding medium for frozen tissue specimens (OCT) in liquid nitrogen. If present, background autofluorescence was subtracted by computer graphics, from the FITC-VT1 B staining.

Methods:

Verotoxin Purification.

Recombinant VT1 [Petric, M. et al., FEMS Microbiol Letts. 41:6-68 (1987)] and VT1 B subunit [Ramotar, K. et al., Biochem. J. 272:805-811 (1990)] were purified by a novel affinity chromatographic technique recently developed [Boulanger, J. et al., Anal Biochem. 217:1-6 (1994)].

Glycolipid Analysis.

Frozen tumor tissue samples were thawed, weighed, homogenized in a minimum volume of PBS and extracted overnight with 20 volumes of 2:1 chloroform:methanol (v/v). The suspension was filtered and the tissue was re-extracted using 10 volumes of 1:1 chloroform: methanol. The combined extracts were partitioned against water [Folch, J., et al., J Biol Chem. 226:497-521 (1957)]. The resulting upper phase was partitioned again against theoretical lower phase. The combined lower phases were dried down and resuspended in 1 ml of 98:2 chloroform:methanol. The samples were then loaded onto silica A columns and washed with 3 column volumes of chloroform and eluted with 10 volumes of 9:1 acetone: methanol. The acetone:methanol fraction containing neutral glycolipids was then dried and resuspended in 0.1-0.5 ml of 2:1 (v/) chloroform: methanol and stored at -20 ° C.

TLC Overlay.

Aliquots of lipid extract equivalent to 10 mg of original tissue were applied to TLC plates and separated in 65:25:4 chloroform:methanol:water(v/v/v). The plates were air dried, and blocked overnight with 1% gelatin in water at 37° C. After rinsing with TBS, the plates were incubated with purified VT1 (1 μg /15 ml TB S), PH I mouse monoclonal anti-VT1 (1 μg/ml in TBS) followed by goat anti-mouse IgG conjugated to horseradish peroxidase (0.5 μg/ml in TBS). The plates were rinsed with TBS after each incubation, and following the final incubation the plates were developed with 4 chloro-l-naphthol [Lingwood, C. A. et al., J Biol Chem. 262:8834-8839 (1987)].

FITC-VI B Subunit Staining of Tumors Section:

Samples of surgically removed ovarian tumors or metastasis were embedded in OCT embedding compound, flash frozen in liquid nitrogen, sectioned and stored at −70° C. Serial 5 μM cryosections of samples were thawed, dried, blocked with BSA and stained with FITC-VT1 B in PBS (0.5 μg/ml) containing 0.1% BSA for 1 hr at room temperature [Lingwood, C. A. et al., Nephron, 66:21-28 (1994)]. Sections were (extensively washed with PBS, mounted with mounting media with antifading agent DABCO, [Krenik, K D., et al., J. Immunol Methods. 117:91-97 (1989)] and observed under incident uv illumination. Adjacent serial sections were stained with hematoxylin/eosin for comparison.

### Example 1

### VT1 Receptor Glycolipid Level in Primary Ovarian Tumors and Metastasis.

Gb₃ is essential for VT1 binding, internalization, and VT-induced cytotoxicity [Waddell, T., et al., Proc. Natl. Acad. Sci. USA 87:7898-7901 (1990)]. The level of expression of Gb₃ in tumors may provide a marker to index potential susceptibility to VT treatment. Gb₃ expression was investigated in different ovarian tumors, metastases and normal ovaries. Altogether, 36 samples, including epithelial ovarian tumors and endometriods cysts and 10 normal ovary controls, were selected for Gb₃ analysis in this study. The presence of Gb₃ was detected by VT1 overlay and quantitated in comparison to a renal Gb₃ standard.

Normal Ovaries.

In the normal ovarian tissue samples, Gb₃ was completely absent in 2 of the 10 samples and was present at concentrations 0.0-0.3 μg per 10 mg tissue in the other 8 samples.

Serous Tumors.

Gb₃ was present in all five serous cyst-type adenomas, ranging in concentration from less than 0.5 μg/10 mg tissue to >2.0 μg/10 mg per tissue. The two cystadenoma samples had significantly higher Gb₃ concentrations (2-3 μg) than the cystadenofibromas samples (Gb₃ concentrations: 0.3-1 μg). Gb₃ expression in the serous adenocarcinomas was more variable. The four serous papillary adenocarcinomas samples that were moderately differentiated had barely detectable Gb₃ whereas those samples which were poorly differentiated had Gb₃ present in higher concentrations (0.5-1.0 μg per 10 mg tissue). The samples from moderately differentiated serous adenocarcinoma tumors whose origin was uncertain(either ovarian or peritoneal) had a Gb₃ concentration of 3 μg per 10 mg tissue equivalent.

Endometrioid Tumors.

Gb₃ was present in concentration ranging from 0.5-1.5 μg per 10 mg tissue equivalent in the four endometrioid cyst samples and endometrioid carcinoma.

Mucinous Tumors.

Gb₃ was detected in ¾ mucinous cystadenomas at low concentrations of 0.1-0.3 μg/10 mg tissue but increased in the mucinous cystadenocarcinoma.

Metastases.

The level of Gb₃ was examined in ovarian metastases to the colon, the small bowel and omentum. A tumor of unknown origin which had metastasized to the ovary was also studied. The expression of Gb₃ was high in all the metastatic (drug resistant) tissues analyzed (even relative to primary ovarian-tumor tissue) ranging from 3-8 μg per 10 mg tissue. Multiple drug resistant primary tumors showed higher Gb₃ content relative to drug sensitive counterparts. In particular, the more slowly migrating isoforms of Gb₃, likely containing shorter fatty acid chains within the lipid moiety, were elevated. These data are summarized in below:

| | Gb3 content | Number of |
|---|---|---|
| Tissue sample | (ug/10 mg) + SD | samples |
| Normal ovary | 0.1 ± 0.1 | 10 |
| Serous tumour | 1.5 ± 0.8 | 5 |
| Mucinous tumour | 0.18 ± 0.17 | 4 |
| Endometrioid tumour | 1.2 ± 0.7 | 5 |
| Metastases | 5.9 ± 2.1 | 6 |

### Example 2

### FITC-VT1B Overlay of Ovarian Tumor and Metastatic Frozen Sections.

Treatment of frozen ovarian or metastatic tumor sections with FITC-VT1 B showed selective toxin binding to the tumor cells. In addition, the lumen of blood vessels within and adjacent to the tumor mass showed extensive VT binding. In general, binding correlated with the levels of Gb₃ extracted from the tissue. Poorly differentiated tumors generally expressed more Gb₃ and showed marked PITC-VT1B binding. Exceptions were noted however for differentiated tumors in which clinical outcome was unexpectedly poor, due to the rapid onset of a multidrug resistant phenotype. In such MDR cases, extensive binding of the toxin to the differentiated tumor cells was seen. Little or no staining of VT1 B was seen in sections of normal ovary. Significant labeling of FITC-VT1B was observed in metastatic tissue, correlating with high Gb₃ content. Ovarian metastases to the colon and small intestine were investigated. Extensive lumenal binding of invading blood vessels was observed and tumor cell foci were selectively bound while stromal cells remained VT unreactive. Necrotic tissue was not stained. VT1 B staining of blood vessels within the normal-bowel or ovary, however, was minimal.

Discussion:

While Gb₃ was barely detectable in normal ovarian tissue samples, the amount of Gb₃ in the tumor specimens was generally increased, suggesting that ovarian cancer cells may be more susceptible to VT than their normal counterparts. There was marked variability of Gb₃ content between different tumor types (endometrioid, mucinous, and serous originating from surface epithelium of the ovary) suggesting differing susceptibility between neoplasm types. Endometrioid tumors had consistently elevated Gb₃ levels while serous tumors were more variable. For the serous papillary adenocarcinomas, Gb₃ content was inversely related to extent of differentiation. While the Gb₃ content was lower for mucinous tumors, tissue section overlay also demonstrated more extensive VT binding to dedifferentiated, as opposed to differentiated, tumor sections. Estimation of Gb₃ content by the overlay of the tissue lipid extract is only semiquantitative at best. Moreover, variation in the normal/tumor cell ratio in a tissue sample may render the analysis of total Gb₃ less informative. Thus the VT tissue section overlay provides the most informative assay of Gb₃ content. Differentiation is part of a mechanism for growth arrest and has great impact on prognosis of ovarian cancer. Poorly differentiated ovarian tumors are far more aggressive with worse prognosis than more differentiated tumors [Baak, J. P. A., et al., J ain Pathol. 39:1340-1346(1986)]. The differentiated tumor which was multidrug resistant showed atypically high Gb₃ content and corresponding intense VT tissue staining. This correlates with the previous observation that MDR ovarian cells lines are hypersensitive to VT and contain more Gb₃ than the drug sensitive parental cell line [Frakas-Himsley, H., et al., Proc Natl Acad Sci. 92:6996-7000 (1995)]. Ovarian tumors deemed to be drug resistant at surgery were analysed for Gb₃ content by toxin overlay of the glycolipid extract.

One of the samples analysed was found to have a less elevated Gb₃ content than expected for MDR. On follow up, it was found that this patient's aggressive tumor showed a surprisingly good response to chemotherapy and therefore was not MDR. Thus analysis of ovarian carcinoma Gb₃ content may provide a method to predict the MDR phenotype (or response to treatment). In this regard, it is of interest to note that the level of glucosyl ceramide, a precursor of Gb₃, has already been proposed as a marker for MDR [Lavie, Y., J Biol Chem. 271:19530-19536 (1996)].

Both these MDR tumors and the MDR ovarian cell lines previously analysed [Farkas-Himsley, H., Proc Natl Acad Sci., 92:6996-7000 (1995)] showed increases in the more slowly migrating forms of Gb₃. This heterogeneity is a function of the lipid moiety which has been implicated in both receptor function and intracellular routing [Lingwood, C. A., Glycoconj J., 13:in press (1996); Pellizzari, A. et al., Biochem. 31:1363-1370 (1992); Kiarash, A., et al., J Biol Chem. 269:11138-11146 (1994)].

Gb₃ expression was elevated in ovarian metastases. VT1 B binding in sections from such tumors showed a clear discrimination between the metastatic tumor cell foci and the background normal stroma. Blood vessels which vascularize the tumor were also highly VT reactive. Both of these findings have significant clinical implications, since the development of drug resistance is the major obstacle in cancer treatment and angiogenesis, which is particularly important in ovarian cancers [Hollingsworth, H. C., et al., Am J Pathol. 147:33-41 (1995)], increases markedly in invasive tumors and metastases. The finding that blood vessels within the normal colon and ovary are not bound by VT suggests that Gb₃ may be specifically up regulated in blood vessels which vascularize tumors.

The fact that the microvasculature of the colon was not toxin reactive is also relevant in considering the role of VT in the etiology of hemorrhagic colitis [(Riley, L. W., Clin. Microbiol. Newsletter 7:47-49 (1985); Riley, L. W., N Engl J Med., 308:681-685 (1983)]. While it is possible that there is heterogeneity in the expression of Gb₃ within the GI microvasculature between individuals, and receptor expression may vary regionally, this result may indicate that endothelial cells of the gastrointestinal tract require an additional signal to induce Gb₃ synthesis, such as we have proposed is required for adult renal endothelial cell Gb₃ synthesis and VT sensitivity[Lingwood, C. A. Nephron, 66:21-28 (1994)], in order to become liable to VT mediated cytotoxicity, following gastrointestinal VTEC infection. In contrast, following IV administration, VT was found to bind effectively to a fraction of the gastrointestinal microvasculature of the rabbit [Richardson S. E. et al., Infect. Immun. 60:4154-4167 (1992)]. A more extensive study of the binding of VT to different regions of the human GI tract will be required to resolve this question.

While the normal function of Gb₃ is not clearly understood, Gb₃ has been implicated in the modification of cell growth parameters. Cell binding of the VT1 B subunit alone can induce apoptosis [Mangeney, M., et al., Cancer Res. 53:5314-5319 (1993)] in B cells and Gb₃ (CD77) containing B cells are prone to apoptosis during B-cell differentiation (Mangeney, M., et al., Eur. J. Immunol. 21:1131-1140 (1991)]. Sensitivity to VT1 is a function of cell cycle and cells at G1/S boundary are particularly sensitive while stationary phase cells are refractory [Pudymaitis, A. et al., J Cell Physiol. 150:632-639 (1992)]. Once internalized by receptor mediated endocytosis [Khine, A. A., et al., J Cell Physiol. 161:319-332 (1994)], Gb₃-bound VT1 can follow a unique pathway of intracellular retrograde transport to the Golgi/ER and nuclear membrane [Khine, A. A., et al., J Cell Physiol. 161:319-332 (1994); Sandvig, K., et al., J Cell Biol. 126:53-64 (1994)]. Gb₃ binding is involved in α-interferon receptor function [Lingwood, C. A., et al., Biochem, J. 283:25-26 (1992); Ghislain, J., et al., J Immunol. 153:3655-3663 (1994)], and in CD 19 signal transduction in germinal center B cells to mediate homotypic adhesion [Maloney, M. D., et al., J Exp Med. 180:191-201(1994)] and apoptosis [Khine, A. A., submitted]. Several cancers have been reported to contain elevated levels of Gb₃ compared to their normal counterparts [Mannori, G., et al., Int J Cancer 45:984-988 (1990); Li, S. -C., et al., Biochem J. 240:925-927 (1986)]. Gb3 has even been proposed as a marker for Burkitts lymphoma [Nudelman, E., Science 220:509 (1983); Murray, L. J., et al., Int. J. Cancer. 36:561-565 (1985)], testicular [Ohyama, C., et al., Int J. Cancer 45:1040-1044 (1990)] and certain germ cell cancers [Wenk, J., et al., Int J Cancer 58:108-115 (1994)]. Human astrocytoma cell lines express high levels of Gb₃ and are highly sensitive to VT1 (or VT1 B subunit) induced apoptosis [Arab, S., et al. Neuropathol Exp Neurol, (in press)]. In KHT sarcoma cell lines, surface Gb₃ expression varied greatly but correlated with metastatic potential [Mannori, G., et al., Int J Cancer 45:984-988 (1990)]. VT has been proposed as a mechanism to target Gb₃ positive nonhodgkin's lymphomas in bone marrow in vitro [LaCasse, E. C., et al., Blood 88:1561-1567 (1996)].

The present studies extend our earlier report that primary malignant ovarian tumors contained higher levels of Gb₃ than normal ovaries [Farkas-Himsley, H., et al., Proc Natl Acad Sci 92:6996-7000 (1995)]. Multi-drug resistant variants of ovarian tumor cell lines were markedly more sensitive to VTs-cytotoxicity than the drug sensitive parental cell line and contained more Gb₃ [Farkas-Himsley, H., et al., Proc Natl Acad Sci 92:6996-7000 (1995)]. This correlates with our present finding that those differentiated ovarian tumor samples which were atypically highly toxin reactive, rapidly became (or were initially) multidrug resistant in vivo. Gb₃ expression and VT binding in differentiated ovarian tumors may thus provide a predictor of poor clinical outcome.

Since histological subtyping of ovarian carcinomas is of limited prognostic value [Baak, J. P. A., J Clin Pathol 39:1340-1346 (1986)], Gb3 expression monitored by VT tissue section overlay can function as a marker of prognosis in ovarian tumors. Moreover, the level of Gb₃ expression was elevated in all of the metastatic samples irrespective of the mechanism of drug resistance of the primary tumor.

VTs are involved in etiology of the hemolytic uremic syndrome, primarily a disease of children under three and elderly [Riley, L. W., N Engl J Med. 308:681-685 (1983)] and hemorrhagic colitis [Griffin, P. W., et al. Epidem Rev. 13:60-98 (1991); Richardson, S. E. et al., Hum Pathol 19:1102-1108 (1988)], both of which are characterized by microangiopathy resulting from endothelial damage to the gastrointestinal and renal vasculature respectively following gastrointestinal infection with VT producing E. coli. Receptors for VT are present in the renal glomeruli of infants but are absent in the glomeruli of adults [Lingwood, C. A., Nephron 66:21-28 (1994)]. Although cultured human renal glomerular endothelial cells are highly toxin sensitive [Obrig, T. G., Recent Advances in Verocytoxin-Producing Escherichia Coli Infections; Elsevier Science B.V. 317-324 (1994)], since such cells are receptor negative in vivo, we have postulated [Lingwood, C. A., Nephron 66:21-28 (1994)] that an additional factor, manifest during the gastrointestinal bacterial infection, e.g., LPS induced cytokines [Tesh, V. L., et al., Infect Immun 62:5085-5094 (1994); Kaye, S. A., et al. Infect Immun. 61:3886-3891 (1993); van der Kar, N. C. J., et al., Blood 85:734-743 (1995)], is responsible for the in vivo induction of renal endothelial Gb₃ synthesis, to account for the incidence of HUS in the elderly following VTEC infection. Similarly, the microvasculature of the colon lacks VT receptors, indicates that a similar induction of Gb₃ synthesis may be necessary for gastrointestinal VT sensitivity following infection with VT producing E. coli. Thus neither renal nor gastrointestinal pathology may pose a threat for VT treatment of cancer patients.

Angiogenesis is essential for growth of both primary and secondary tumors. VT treatment may offer duel targeting of both the tumor and its vascular supply. Inhibition of angiogenesis provides a novel and more general approach for treating metastases by manipulation of the host microenvironment. Endothelial cells in tumor blood vessels divide rapidly, whereas those in normal tissues do not [Folkman, T., Nature Med 1:27-31 (1995)]. This may also relate to the selective VT staining of pediatric, as opposed to adult, renal glomeruli [Lingwood, C. A., Nephron 66:21-28 (1994)]. Extensive staining of blood vessels which vasculerize the tumor was observed irrespective of the chemical Gb₃ status of the tumor itself. Thus VT has an antiangiogenic effect even for Gb₃ negative tumors.

Interferon-α is the most widely studied inhibitor of angiogenesis and chronic daily administration of low dose interferon α has been shown to induce complete regression of life threatening hemangiomas in infants [Ezekowitz, R. A., et al., N Engl J Med 326:1456-1463 (1992)] and highly vascular Kaposi's sarcoma [Real, F. X., et al., J Clin Oncol 4:544-551 (1986)]. Gb₃ appears to be involved in α₂-interferon signal transduction [Ghislain, J., et al., J Immunol 153:3655-3663 (1994); Cohen, A. et al., J. Biol. Chem. 262:17088-17099 (1987)], resulting from the sequence similarity of the α₂-interferon receptor and the VT B subunit [Lingwood, C. A., et al., Biochem J. 283:25-26 (1992)]. Interestingly, the α2-interferon receptor itself has been found to show antineoplastic activity [Colamonici, O., et al., J Biol Chem 269 (1994)].

Genotoxic insults such as radiation and chemotherapy are known to induce apoptosis [Strasser, A., et al., Cell 79:189-192 (1994)]. In fact, apoptosis has been recognized as the major mechanism in the action of many chemotherapeutic agents [Barry, M. Biochem Pharma 40:2353-2362 (1990)]. However, the over expression of Bcl 2 renders the tumors resistance to the apoptotic activity of the anti-cancer drugs [Wang, Y., et al., Oncogene 8:3427-3432 (1993)]. This is of considerable therapeutic importance when tackling the thorny problem of drug resistance. Since multidrug resistant tumors and cell lines are oversensitive to VT, this pharmacological attack may prove to be of great importance in circumventing the problem of drug resistance. VT effectively induced apoptosis in drug resistance ovarian cell lines over expressing P-glycoprotein [Farkas-Himsley, H., et al., Proc Natl Acad Sci. 92:6996-7000 (1995)].

The inhibition of tumor-induced angiogenesis, coupled with the increased levels of Gb₃ found in ovarian tumor cells and metastases, the most challenging aspect of cancer, indicate that VT treatment offers a promising alternative for Gb₃ containing tumors.

### Example 3

### Human Tissue Cross Reactivity of Verotoxin with Normal and Neoplastic Human Tissue

Materials and Methods:

In order to detect binding, the test article (VT) was applied to selected normal and neoplastic human tissues at a concentration of 20 ng/mL. Tissues that had been obtained previously via autopsy or surgical biopsy were embedded in Tissue-Tek® O.C.T. medium, frozen on dry ice and stored in sealed plastic bags below -70° C. until staining and fixed for 10 seconds in 10% neutral buffered formalin at room temperature just prior to staining. Cryosections of Daudi cells were used as positive control and cryosections of VT 500 cells were used as negative control tissue.

In two follow-up experiments, VT was added to additional tissues and increased concentration. The first included kidney (two adults and one infant) and cerebellum cryosections at a concentration of 50 ng/mL using unfixed sections and sections that had been fixed in 10% neutral buffered formalin as described above. The purpose of this experiment was to determine any possible effects of the fixation on the binding and to determine whether increased concentration of the VT would detect additional ligands that were not observed at 20 ng/mL. The second experiment was performed on unfixed tissues using 50 ng/mL and 200 ng/mL of VT. To reveal binding sites in neoplastic tissues with higher test material concentrations samples of astrocytoma and ovary carcinoma (two donors each) were evaluated for binding. The HT 168 astrocytoma was classified as low grade astrocytoma with low pleomorphism, cellularity and no mitotic figures. Astrocytoma HT 196 was moderately pleomorphic and cellular with few mitotic figures. The ovary carcinoma HT 162 was described as poorly differentiated mucinous adenocarcinoma with goblet cell enteric differentiation. HT 163 ovary carcinoma was a metastatic papillary serous cystic adenocarcinoma with ovary as site of tumor origin.

Materials

1. Test article VT I, Lot No. PTI, at a protein concentration of 1 mg/mL, Select Therapeutics, Cheshire, Conn. PAI No. A1621.

2. Anti-VT 1 B-subunit, Lot No. not supplied by sponsor, at a protein concentration of 1.6 mg/mL, Select Therapeutics, Cheshire, Conn., PAI No. A1618.

3. Glucose oxidase, Sigma, St. Louis, Mo., Lot No. 46F39031.

4. Human gamma globulin, Sigma, St. Louis, Mo, Lot No. 76H9315.

5. Avidin-biotin-peroxidase kit (ABC Elite), Vector Labs, Burlingame, Calif., Lot No. PK-6100, PAI No. K301.

6. 3,3′-Diaminobenzidine (DAB). Sigma Fast Tablets, Sigma, St. Louis, Mo., Lot No. 17H8927.

7. β-D(+) Glucose, Sigma, St. Louis, Mo., Lot No. 35H0626.

8. Bovine serum albumin (BSA), Sigma, St. Louis, Mo., Lot No. 77H0699.

9. Casein, Sigma, St. Louis, Mo., Lot No. 16H0685.

10. Sodium chloride, Sigma, St. Louis, Mo., Lot No. 47H0203.

11. Avidin-biotin blocking kit, Vector Labs, Burlingame, Calif., Lot No. 11029.

12. Sodium phosphate, dibasic, Sigma, St. Louis, Mo., Lot No. 76H1024.

13. Potassium phosphate, monobasic, Mallinckrodt, Paris, Ky., Lot No. 7100KLJS.

14. Normal goat serum, Vector Labs, Burlingame, Calif., Lot No. 10728.

15. Biotinylated Goat anti-human IgG, Fcg fragment specific, Jackson ImmunoResearch Laboratories, inc., West Grove, Pa., Lot No. 35140, PAI No. A1275.

16. Sodium azide, Sigma, St. Louis, Mo., Lot No. 46H0306.

17. Human tissues, National Disease Research Institute, Philadelphia, Pa.

18. Positive control Daudi cells, Lot No. not provided by Sponsor, Select Therapeutics, Cheshire, Conn., (expiration date not provided by sponsor).

19. Negative control VT 500 cells, Lot No. not provided by Sponsor, Select Therapeutics, Cheshire, Conn., (expiration date not provided by Sponsor).

20. Biotinylated goat anti-mouse IgG (H+L), Jackson ImmunoResearch Laboratories, Inc., West Grove, Pa., Lot No. 35411, PAI No. A1606.

Immunoperoxidase Staining Method:

On the day of staining, the cryosections of normal and neoplastic human tissues, positive control Daudi cells, and negative control VT500 cells were fixed in 10% formalin for 10 seconds at room temperature (experiment 1 and 2) or were used without fixation (experiment 3). Next, endogenous peroxidase activity was quenched by incubating the tissues in a solution containing sodium azide (1 mM), glucose (10 mM), and glucose oxidase (1 U/mL) for 60 minutes at 35° C. Next, non-specific binding of reagents was blocked by incubation with sequential changes of avidin and biotin solutions (15 minutes each) and a protein solution (0.5% casein, 1% BSA, and 1.5% normal goat serum in phosphate-buffered saline [PBS], 20 minutes). Then VT I was applied to the tissues for 60 minutes at concentrations of 20 ng/mL. (Run 1, dilution of 1:50,000), 50 ng/mL (Run 3 and 4, dilution of 1:20,000), and 200 ng/mL (Run 4, dilution of 1:5,000). The primary antibody was eliminated from another slide (assay control). Following application of the test article, anti-VT I was added to the tissue sections for 30 minutes at a 1:1600 dilution. Next, biotinylated goat anti-mouse antibody was applied for 30 minutes at a dilution of 1:500. Subsequently, all slides were reacted for 30 minutes with ABC Elite reagent. DAB was applied for 4 minutes as substrate for the peroxidase reaction. Slides were counterstained with hematoxylin, dehydrated and coverslipped for light microscopic evaluation. Staining intensity was graded semi-quantitatively using the following scale: +/− (equivocal), 1+ (weak), 2+ (moderate), 3+ (strong), 4+ (intense), Neg (negative).

The complete study file and attendant study materials are retained in the PAI Archive, Frederick, MD 21701.

Results:

Positive and Negative Controls:

The results are included in the tables. The test article reacted intensely with the positive control Daudi cells and did not bind to negative control VT500 cells at 20 and 50 ng/ml. On occasion VT 500 cells stained at 200 ng/ml.

Human Test Tissues:

The results are summarized in Table I for the 20 ng/mL concentration. Specific binding of the test article at 20 ng/mL was observed to endothelium of many but not all capillaries and small vessels of the following normal and neoplastic human tissues: lymph node [two of two donors tested positive (2/2)], cerebellum (2/3), cerebrum (2/2), uterine carcinoma, (2/2), breast carcinoma (2/2), ovarian carcinoma (2/2), and cervical carcinoma (1/1). Binding to vascular smooth muscle was observed in the following tissues: cerebrum (1/2), uterine carcinoma (1/2), and ovarian carcinoma (1/2). Specific binding was further observed to occasional epithelial cells, presumed neoplastic in one uterine carcinoma (HT178) and mononuclear cell aggregates in one lymph node (HT341). Granular staining of unidentified interstitial cells was noted in the section of skeletal muscle In the kidney sections, there was binding only to the periphery of the renal sections which were composed of distal nephron epithelium. This was interpreted to be edge artifact, a common artifact of immunohistochemistry. With this concentration no binding was noted in the following normal and neoplastic tissues: cerebral neurophil, (2/2), kidney (except as indicated above), and astrocytoma (2/2).

Using 50 ng/mL of the test material (results in Tables 2 and 3), binding to endothelium of small vessels was observed in the cerebellum (3/3) and the kidneys (3/3). In addition, staining was observed on the tubular epithelium of distal nephrons of the adult kidneys (2/2) and both proximal and distal nephron tubular epithelium of the infant kidney (1/1). Focal patchy staining of vascular smooth msucle was observed in 2/3 cerebellar sections. Staining was generally slightly more intense in the unfixed sections compared to the formalin-fixed sections, indicating slight interference with binding by the fixation procedure. Increased staining of the tissues was observed with VT at a concentration of 50 ng/mL compared to 20 ng/mL suggesting that although binding was observed at the lower concentration, the high concentration was needed for ligand saturation.

200 ng/mL and 50 ng/mL of VT were tested on two astrocytomas and ovary carcinomas, each. The astrocytomas were of two donors previously tested, no revealing any positive staining with 20 ng/mL of VT. The ovary carcinomas had not been tested before. Binding to vascular endothelium of some but not all small and medium sized vessels was consistently seen in all sections. Vascular smooth muscle was positive in one (HT 168) of the astrocytoma sections, only. The majority of neoplastic cells of both ovary carcinomas were negative for binding with VT. However, staining of selected neoplastic epithelial cells was observed in both ovary carcinomas. In one sample (HT163) binding was consistently seen with neoplastic epithelium lining tubular spaces. Most of the positive immune reaction was located in the apical cytoplasm of these cells. Occasional binding was also present scattered throughout the tissue to individual neoplastic epithelial cells. Neoplastic astrocytes comprising the astrocytomas tested were not specifically bound by VT. Staining of unidentifiable mononuclear cells in the astrocytoma samples was considered nonspecific background since staining was equally present in the assay control.

Summary:

The test article VT I, Shiga-like toxin produced by strains of Escherichia Coli was applied to cryosections of selected normal and neoplastic human tissues at protein concentrations of 20, 50 and 200 ng/mL to determine binding with these tissues. The effect of formalin fixation versus nonfixation of selected tissue was also compared for the 560 ng/mL.

For the 20 ng/mL run, specific binding of the test article was observed to vascular endothelium in 15 out of 19 tested normal and neoplastic human tissues (lymph node, cerebellum, cerebrum, skeletal muscle, lymph node, uterine carcinoma, breast carcinoma, ovarian carcinoma, and cervical carcinoma). Reaction with vascular smooth muscle was observed in 3 test tissues (cerebrum, uterine carcinoma and ovarian carcinoma). Staining with rare neoplastic epithelial cells was observed in a uterine carcinoma. There was specific binding to mononuclear cell aggregates in one lymph node and granular staining of unidentified interstitial cells in skeletal muscle. In the kidney, binding was observed in distal nephron epithelium but this was conisdered an artifact (edge artifact and was repeated at 50 ng/mL. At 20 ng/mL, no binding was noted in the following normal and neoplastic tissues: cerebrum (neuropil), kidney (except as indicated above0, and astrocytoma.

Replicate samples of cerebellum and kidney (two adult and one infant) were stained again with VT at 50 ng/mL. This concentration was chosen because no background staining was observed in preliminary studies and it represented a cocnentration that might produce enhanced signal on target ligands. In addition, the tissues were stained in duplicate; one replicate was not fixed and the other replicate was fixed for 10 seconds in 10% neutral buffered formalin. The test article bound to vascular endothelium in all sections of cerebellum and kidney. In addition, renal tubular epithelium of the distal nephron stained from the sections of adult kidney and both proximal and distal neprhon epithelium stained in the sections of juvenile kidney. binding to vascualr smooth muscle was seen in two cerebellar sections. Staining was slightly enhanced in cryosections which were not fixed.

In a third experiment two VT concentrations were tested. 50 ng/mL and 200 ng/mL to reveal binding sites in neoplastic tissues. Astrocytoma sections previously tested with lower concentrations of VT (20 ng/mL) and ovary

Discussion:

The observed binding of VT mainly to capillary vascular endothelium is consistent with findings of others who identified the tissue distribution of CD77 with means of a variety of monoclonal antibodies (Oosterwijk, et al. 1991; Kasai, et al., 1985, Pallensen, et al., 1987). Vascular smooth muscle binding, and reactions with selected neoplastic epithelia were also reported in these citations. Smooth muscle binding with VT was noted only very selectively in the present study. Staining of renal glomeruli and tubules and many other sites within lymphoid tissues and neoplasms were additionally identified as location of the CD77 antigen by these authors. With increase in the test article concentration VT binding was detectable in renal sections and ovary carcinomas tested in this study. However, the astrocytomas available for testing did not reveal specific VT binding. A reason for this could be a difference of the antigenic moities recognized by the monoclonal antibodies referenced in the literature and the test substance. However, an increase of the VT concentration significantly increased the number of identifiable binding sites present in normal and neoplastic tissues. This could imply potential loss of antigenic sites due to preparative techniques. In addition, both astrocytomas were rather well differentiated contrasting the ovary carcinomas tested, which were poorly differentiated. The degree of loss of differentiation may correlate with the expression of VT binding sites.

TABLE 1
Verotoxin In Tissue
Formalin Fixed Tissue

| | | VEROTOXIN | ASSAY |
|---|---|---|---|
| TISSUE | SOURCE | 20 ng/ml | CONTROL |
| Positive Control | | | |
| Daudi Cells | | 2-4+ | Neg |
| Negative Control | | | |
| Vt500 Cells | | Neg | Neg |
| Cerebellum | | | |
| Vascular endothelium | HT 305 | 1-2+ | Neg |
| (primarily capillary) | | | |
| Cerebellum | | | |
| Vascular endothelium | HT 410 | Neg | Neg |
| (primarily capillary) | | | |
| Cerebellum | | | |
| Vascular endothelium | HT 446 | 2-3+ | Neg |
| (primarily capillary | | | |
| Cerebrum | | | |
| Vascular endothelium | HT 410 | 2-3+ | Neg |
| (primarily capillary) | | | |
| (vascular smooth muscle) | | 2-3+ | Neg |
| Cerebrum | | | |
| Vascular endothelium | HT 476 | Neg | Neg |
| Kidney | HT 085 | Neg | Neg |
| Kidney | HT 089 | Neg | Neg |
| Lymph Node | | | |
| Vascular endothelium | HT 329 | 2-3+ | Neg |
| Lymph Node | | | |
| Vascular endothelium | HT 341 | 3-4+ | Neg |
| Mononuclear cells of focal | | 1-2+ | Neg |
| aggregates in cortex | | | |
| Skeletal Muscle | | | |
| Interstitial cells (unidentified) | HT 029 | 2-3+ | Neg |
| with granular staining | | | |
| Astrocytoma | HT 168 | Neg | Neg |
| Astrocytoma | HT 196 | Neg | Neg |
| Breast Carcinoma | | | |
| Vascular endothelium | HT 145 | 2-4+ | Neg |
| Breast Carcinoma | | | |
| Vascular endothelium | HT 308 | 3-4+ | Neg |
| Cervix Carcinoma | | | |
| Vascular endothelium | HT 255 | 3-4+ | Neg |
| Ovary Carcinoma | | | Neg |
| Vascular endothelium | HT 172 | 2-3+ | Neg |
| Smooth musculature | | 2-3− | Neg |
| Ovary Carcinoma | | | |
| Vascular endothelium cells | HT 139 | 3-4+ | Neg |
| Uterine Carcinoma | | | Neg |
| Vascular endothelium | HT 139 | 2-3+ | Neg |
| Uterine Carcinoma | | | |
| Vascular endothelium | HT 178 | 3-4+ | Neg |
| Vascular smooth muscle | | 2-4+ | Neg |
| Rare neoplastic epithelial cells | | 3-4+ | Neg |

TABLE 2
Verotoxin In Tissue
Formalin Fixed Tissue

| | | VEROTOXIN | ASSAY |
|---|---|---|---|
| TISSUE | SOURCE | 50 ng/ml | CONTROL |
| Positive Control | | | |
| Daudi cells | | 2-4+ | Neg |
| Negative Control | | | |
| VT500 Cells | | Neg | Neg |
| Cerebellum | | | |
| Vascular endothelium | HT 305 | 2-3+ | Neg |
| (primarily capillary) | | | |
| Cerebellum | | | |
| Vascular endothelium | HT-140 | 1-2+ | Neg |
| (primarily capillary) | | | |
| Vascular Smooth Muscle | | 2-4+ | Neg |
| Cerebellum | | | |
| Vascular endothelium | HT 439 | 3-4+ | Neg |
| (primarily capillary | | | |
| Vascular Smooth Muscle | | Neg | Neg |
| Kidney (Infant) | | | |
| Tubular epithelium | HT 087 | 2-4+ | Neg |
| (proximal and distal nephrons | | | |
| Vascular endothelium | | 2-3+ | Neg |
| (patchy) | | | |
| Kidney (Adult) | | | |
| Tubular epithelium | HT 089 | 3-4+ | Neg |
| (distal nephrons) | | | |
| Vascular endothelium | | 1-2+ | Neg |
| (patchy) | | | |
| Kidney (Adult) | | | |
| Tubular epithelium | HT 120 | 1-3+ | Neg |
| (distal nephrons) | | | |
| Vascular endothelium | | 2-3+ | Neg |
| (patchy) | | | |

TABLE 3
Verotoxin In Tissue
Unfixed Tissue

| | | VEROTOXIN | ASSAY |
|---|---|---|---|
| TISSUE | SOURCE | 50 ng/ml | CONTROL |
| Positive Control | | | |
| Daudi Cells | | 2-4+ | Neg |
| Negative Control | | | |
| VT500 Cells | | Neg | Neg |
| Cerebellum | | | |
| Vascular endothelium | HT 305 | 3+ | Neg |
| (primarily capillaries) | | | |
| Cerebellum | | | |
| Vascular endothelium | HT 410 | 3-4+ | Neg |
| (primarily capillaries) | | | |
| Vascular Smooth Muscle | | 2-3+ | |
| Cerebellum | | | |
| Vascular endothelium | HT 439 | 3-4+ | Neg |
| (primarily capillaries) | | | |
| Vascular Smooth Muscle | | +/− | Neg |
| Kidney (Infant) | | | |
| Tubular epithelium | HT 087 | 3-4+ | Neg |
| (proximal and distal nephron) | | | |
| Vascular endothelium | | 3-4+ | Neg |
| (patchy) | | | |
| Kidney (adult) | | | |
| Tubular epithelium | HT 089 | 2-4+ | Neg |
| (distal nephron) | | | |
| Vascular endothelium | | 3-4+ | Neg |
| (patchy) | | | |
| Kidney (adult) | | | |
| Tubular epithelium | HT 120 | 3-4+ | Neg |
| (distal nephron) | | | |
| Vascular endothelium | | 3-4+ | Neg |
| (patchy) | | | |

TABLE 4
Verotoxin In Tissue
Cryosections

| | | VERO- | VERO- | |
|---|---|---|---|---|
| | | TOXIN | TOXIN | ASSAY |
| TISSUE | SOURCE | 200 ng/ml | 50 ng/m | CONTROL |
| Positive Control | | | | |
| Daudi Cells | | 3-4+ | 3-4+ | Neg |
| Negative Control | | | | |
| VT500 Cells | | Neg | Neg | Neg |
| Astrocytoma | | | | |
| Vascular endothelium | HT 168 | 2-3+ | 2+ | Neg |
| Vascular Smooth | | 2-3+ | 2+ | Neg |
| Muscle | | Neg | Neg | 2+ |
| Mononuclear cells | | | | |
| (perivascular neuropile) | | | | |
| Astrocytoma | | | | |
| Vascular endothelium | HT 196 | ±−1+ | ± | Neg |
| Mononuclear cells | | 2-3+ | 2+ | 3+ |
| (Glial cells, neuropile) | | | | |
| Ovary Carcinoma | | | | |
| Vascular endothelium | HT 162 | 3+ | 2+ | Neg |
| Ovarian adenocarcinoma | | 2-3+ | 2-3+ | Neg |
| cells | | (tubular, | | |
| | | apical, | | |
| | | cytoplasm; | | |
| | | & single | | |
| | | cells) | | |
| Ovary Carcinoma | | | | |
| Vascular endothelium | HT-163 | 3+ | 2-3+ | Neg |
| cells | | 2-3+ | 2-3+ | Neg |
| Ovarian adenocarcinoma | | (tubular, | | |
| cells | | apical | | |
| | | cytoplasm; | | |
| | | & single | | |
| | | cells) | | |

### Example 4

### Verotoxin Induces Apoptosis and the Complete, Rapid, Long Term Elimination of Human Astrocytoma Xenografts in Nude Mice

Despite the involvement of VTs in clinical disease, we have proposed [Farkas-Himsley, H. et al. Proc Natl Acad Sci 92:6996-7000 (1995); Arab, S. et al. C Oncol Res 9:553-563 (1997)] that VT provides a viable novel approach to the treatment of cancer. We have extended our studies to examine the effect of VT 1 on astrocytoma cell growth in an animal model.

Astrocytoma Tumor Regression.

Rapid tumor regression was observed following a single i.t. injections (doses of 8 μg/kg and 4 μg/kg VTI) of VT1 into nude mice bearing a subcutaneous human astrocytoma tumor derived from the SK 539 astrocytoma cell line. The regression was biphasic with >50% reduction in tumor size within 48 hr and complete regression of tumor within 10-15 days. For the smallest tumors (50 mm diam) the tumor was eliminated by day 7 post VT1 treatment. No tumor reoccurrence was observed within the time frame of the experiment (60 days). The body weight of the mouse increased coincident with tumor regression.

In astrocytoma tumor bearing mice injected with heat-inactivated VT 1, the tumor nearly doubled in size within 30 days. This continued tumor growth was accompanied with severe overall body weight loss.

Mechanism of Cell Death in Tumors Treated with the VT1.

The level of apoptosis within the tumor xenograft 24 hours following VT1 injection was determined by TUNEL staining. In H & E tumor sections, prominent nuclei with irregular membrane and irregular chromatin, abnormal mitotic figures, and tripolar spindle are distinct characteristic features of malignant cells. Interestingly, a high level of angiogenesis is clearly demonstrated in these tumors. Most of the astrocytoma nuclei are stained by the TUNEL procedure, showing significant VT1-induced apoptosis. The nuclei of cells within the tumor vasculature are also intensely stained, indicating that the toxin has targeted the invading blood vessels (mouse) in addition to the tumor xenograft. The apoptotic cells detected by TUNEL assay were verified using an in situ nick translation method.

FITC-VT1B overlay of Human Brain Tumor Frozen Sections.

Preliminary screening of a few primary human astrocytomas was performed to confirm that the VT1 sensitivity of SF 539 cells is clinically relevant. Treatment of frozen tumor sections from surgically removed primary human astrocytoma tumors with FITC-VT1B showered selective, extensive toxin binding to the tumor in high grade malignant glioblastoma. Blood vessels, particularly their lumen, within these tumors were significantly stained with FITC-VT1B. A pediatric low grade astrocytoma tumor showed only low level FITC-VT1B binding.

Discussion:

Intratumor VT1 injection resulted in the total regression of astrocytoma xenografts in vivo, without apparent side effect. All treated mice remained tumor free for more than 50 days post VT1 treatment when they were sacrificed. Efficacy of this nature has not been previously reported. Apoptosis was induced in both tumor cells and microvascular (endothelial) cells within the tumor mass. These data correlate with the expression of the VT receptor in glioblastoma multiform tumor and its vasculature.

In light of the involvement of VT1 in human disease, is it possible to consider VT1 as a candidate antineoplastic? HUS is primarily a renal angiopathy of very young children (when Gb₃ is expressed in renal glomeruli [Lingwood, C. A. Nephron 66:21-28 (1994)]) and the elderly [Carter, A. O., et al. N Engl J Med 317:1496-1500 (1987)] (when glomerular Gb₃ expression is lacking [Lingwood, C. A. Nephron 66:21-28 (1994)]) following gastrointestinal VTEC infection. HUS is a rare occurrence in the general adult population. We have argued [Arab, S. et al., C. Oncol Res 9:553-563 (1997)] that HUS in the elderly may not be the result of acute toxin action, but that a prior endothelial cell activation step, perhaps cytokine mediated, may be involved, in which the synthesis of Gb₃ within the renal microvasculature is induced. This premise is based on the known cytokine-mediated stimulation of endothelial Gb₃ synthesis in vitro [van der Kar, N. C. A. J., Blood 80:2755-2764 (1992); Louise, C. B. and Obrig, T. G. Inf Imm. 60:1536-1543 (1992)] and the postulated role of monocytes [Ramegowda, B. and Tesh, V. L. Infect Immun 64:1173-1180 (1996)] in the generation of such cytokines in HUS and HC. Cytokine production might be initiated by gastrointestinal VTEC-generated LPS [Louise, C. B. and Obrig, T. G. Inf. Imm. 60:1536-1543 (1992)] or by direct VT1 stimulation [Tesh, V. L. et al., Infect Immun 62:5085-5094 (1994); van Setten, P et al., Blood 88:174-183 (1996)]. In either case, renal endothelial sensitization might occur only after several days, providing a window which could permit acute VT1 tumor therapy. Similarly although CNS involvement may be apparent late in severe cases of HUS [2−], cultured human cerebral microvascular endothelial cells are not sensitive to VT [Arab, S., et al., J Neuro Oncol (in press)], again suggesting that additional factors during VTEC infection may sensitize these cells in vivo. For the elderly, in the absence of such an infection and for the adult population in general, VT1 antineoplastic treatment might show limited pathological side effects.

We have shown that the treatment of some human astrocytoma cell lines will induce rapid apoptosis in these cells [Arab, S., et al., J Neuro Oncol (in press)]. Disruption of the nuclear morphology was observed as soon as 90 minutes after toxin addition. Typically, induction of apoptosis in other systems requires 18 hours before morphological evidence of the induction is apparent [Falcieri, E. et al., Scan Microscopy 8:653-666 (1994); Anderson, K. M. et al. Scanning Microscopy 8:675-686 (1994)]. This implies that VT affects a component far downstream in the apoptotic pathway.

Astrocytomas, arising from astrocytes, constitute the majority of primary brain tumors and are the most common gliomas [Thapar, K. et al. Brain Tumors (eds. Kaye, A. & Laws, E.), pp 69-98 (1995)]. The median survival for patients with glioblastoma multiform, the most malignant form of astrocytoma, is approximately 12 months. In this context, it is imperative that new therapeutic strategies continue to be explored for malignant astrocytomas.

Sensitivity to VT1 and toxin/cell binding varies as a function of cell growth and cell cycle progression [Pudymaitis, A. & Lingwood, C. A. J Cell Physiol 150:632-639 (1992)]. Cells at the G1/S boundary are particularly sensitive while stationary phase cells are refractory [Pudymaitis, A. & Lingwood, C. A. J Cell Physiol 150:632-639 (1992)]. Ligation of Gb₃ alone has been shown to induce apoptosis [Mangeney, M. et al. Cancer Res 53:5314-5319 (1993); Taga, S., et al. Blood 90:2757-2767]. These findings are consistent with a role for Gb₃ in growth control. Elevated levels of Gb₃ have been associated with several human tumors and proposed as a marker in some cases [Wenk, J. et al., Int J Cancer 58:108-115 (1994); Li, S. -C. et al., Biochem J 240:925-927 (1986); Wiels, J. et al., Proc Nat Acad Sci (Wash.) 78:6485-6488 (1981)]. We have found that Gb₃ is elevated in primary ovarian tumors and particularly their metastases [Arab, S. et al., Oncol Res 9:553-563 (1997)]. As with astrocytomas, the blood vasculature to ovarian carcinomas is VT reactive. Gb₃ is particularly elevated in multidrug resistant ovarian tumors [Arab, S. et al., Oncol Res 9:553-563 (1997)]. Ovarian carcinoma cell lines selected for multiple drug resistance in vitro were 5000 fold more sensitive to VT than the parental cell line [Farkas-Himsley, H. et al. Proc Natl Acad Sci 92:6996-7000 (1995)]. It is of interest to note that the astrocytoma cell line SF 539, used in the present study, was derived from a patient who had undergone two resections, irradiation and multiple drug chemotherapy [Rutka, J. et al., Cancer Res 46:5893-5902 (1986)] and was therefore also drug resistant. The poor VT staining of the low grade astrocytoma may be a further indication of a relationship between agressivity and VT sensitivity.

We have proposed that GB₃ plays a role in α₂ interferon signaling [Cohen, A. et al., J. Biol. Chem. 262:17088-17099 (1987)]. The α₂ interferon receptor was subsequently found to show sequence similarity to the VT B subunit [Lingwood, C. A. and Yiu, S. C. K. Biochem J. 283:25-26 (1992)] and bind galabiose containing glycolipids [Ghislain, J., et al. J Interfer Res 12:sl 14 (1992)]. Both α₂ interferon [Sexl, V. et al. Clin Invest 72:317-320 (1994)], and the α₂ interferon receptor itself [Colamonici, 0. et al., J Biol Chem 269:27275-27279 (1994)], have antineoplastic activity.

Indeed, both astrocytomas [Buckner, J. et al. J Neurosurg 82:430-435 (1995)] and ovarian carcinomas [Cherchi, P. et al J Gynecol Obst Biol Reprod 25:101-102 (1996)] are sensitive to α2 interferon. Angiogenesis is a prominent feature of aggressive astrocytoma and an essential feature of the histological diagnosis and grading of such tumors [Burger, P. C. et al., Brain Tumors 193-437 (1991)]. Histological examination and TUNEL assay of VT1-treated tumor sections showed that the mechanism of action of VT1 in tumor regression is apoptosis induction. Most of the astrocytoma nuclei within the tumor are stained by TUNEL. In addition, the nuclei of endothelial cells of the blood vessels within the tumor mass were heavily stained, indicating that the VT1 was significantly targeted to the blood vessels as well as the tumor per se. This is consistent with the VT1 staining of the tumor vasculature in the primary glioblastoma sections. This antiangiogenic effect of VT1 by itself, would be significant in VT1 therapy of astrocytoma. Our finding of similar VT1 staining of ovarian carcinoma vasculature [Arab, S. et al. Oncol Res 9:553-563 (1997)] suggests that VT1 sensitivity of tumor neoangiogenesis may be a common aspect of VT1 antineoplastic activity. Regulation of angiogenesis is an attractive approach to cancer treatment [Boehm, T. et al., Nature 390:404-407 (1997)]. VT however, has the added advantage that it shows a bona fide anti-neoplastic effect in addition to antiangiogenesis. This, compounded with the finding that drug resistant tumor cells may be preferentially VT sensitive, would make VT almost ideally suited as an antineoplastic.

The sensitivity of astrocytoma xeonografts to VT1 in vivo adds further support to our contention that judicious use of VT can provide the basis of a new approach to the treatment of Gb₃-expressing human neoplasias.

### Equivalents

Those skilled in the art will recognize, or be able to ascertain using no more than routine experimentation, numerous equivalents to the specific procedures described herein. Such equivalents are considered to be within the scope of this invention and are covered by the following claims.

The contents of all publications, issued patents, pending patent applications, and published patent applications cited herein are hereby incorporated by reference.

Other embodiments are within the following claims.

What is claimed is:

1. A method for inhibiting angiogenesis, the method comprising administering an effective amount of an anti-angiogenic agent that binds Gb₃, such that angiogenesis is inhibited.

2. The method of claim 1, wherein the tissue is a tumor.

3. The method of claim 2, wherein the tumor is a cancer.

4. The method of claim 3, wherein the cancer is selected from the group consisting of breast cancer and ovarian cancer.

5. The method of claim 1, wherein the anti-angiogenic agent is a verotoxin.

6. The method of claim 5, wherein the verotoxin is verotoxin 1.

7. The method of claim 5, wherein the verotoxin is verotoxin 1 B-subunit.

8. The method of claim 5, wherein the verotoxin is verotoxin 2.

9 The method of claim 5, wherein the verotoxin is verotoxin 2c.

10. A method for treating a drug-resistant tumor, the method comprising administering to a subject in need thereof an effective amount of a verotoxin, such that the drug-resistant tumor is treated.

11. The method of claim 10, wherein the drug-resistant tumor is a cancer.

12. The method of claim 11, wherein the cancer is selected from the group consisting of breast cancer, testicular cancer, and ovarian cancer.

13. The method of claim 10, wherein the verotoxin is selected from the group consisting of verotoxin 1, verotoxin 1 B-subunit, verotoxin 2, and verotoxin 2c.

14. The method of claim 10, wherein the verotoxin is administered in a pharmaceutically-acceptable carrier.

15. A method for visualizing a blood vessel, the method comprising contacting the blood vessel with verotoxin such that verotoxin binds to the blood vessel; and
visualizing verotoxin bound to the blood vessel, such that the blood vessel is visualized.

16. The method of claim 15, wherein the visualizing step comprises contacting the bound verotoxin with a verotoxin-binding antibody.

17. The method of claim 16, wherein the verotoxin-binding antibody is labeled.

18. The method of claim 17, wherein the verotoxin-binding antibody is labeled with FITC.

19. The method of claim 15, wherein the verotoxin is selected from the group consisting of verotoxin 1, verotoxin 1 B-subunit, verotoxin 2, and verotoxin 2c.

20. A method for determining whether a tumor is multi-drug resistant, the method comprising:
determining the amount of Gb₃ in a tumor sample;
comparing the amount of Gb₃ in the tumor sample with a preselected value;
thereby determining whether the tumor is multi-drug resistant.
